(19) United States
(12) Patent Application Publication
(10) Pub. No.: US 2001/0036634 A1
(43) Pub. Date: Nov. 1, 2001

(54) Lateral flow PCR with amplicon concentration and detection

(76) Inventors: Timothy Chow, Norwich, VT (US); Timothy Allan Brown, Redwood City, CA (US)

Correspondence Address: CLARK & ELBING LLP, 176 FEDERAL STREET, BOSTON, MA, 02110-2214, US

(21) Appl. No.: 09/801,434

(22) Filed: Mar. 7, 2001

Related U.S. Application Data
(60) Provisional application No. 60/187,919, filed on Mar. 8, 2000.

(51) Int. Cl. C12Q 1/68; C12P 19/34; C12M 1/34

(52) U.S. Cl. 435/6; 435/91.1; 435/287.2

(57) ABSTRACT

The present invention provides an apparatus and method for performing nucleic acid amplification (PCR), concentration and detection on a porous membrane. The apparatus of the present invention is of low cost and is amenable to use in the clinic for rapid and facile analysis of patient samples.

### CROSS REFERENCE TO RELATED APPLICATIONS

This application claims priority from U.S. Provisional Patent Application No. 60/187,919, filed on Mar. 8, 2000.

### BACKGROUND OF THE INVENTION

Since its discovery in 1983, the polymerase chain reaction (PCR) has revolutionized nucleic acid analysis and molecular biology research. However, use of PCR in clinical laboratories is limited due to the amount of labor and skill required for sample preparation, amplification, and detection. In addition, the length of time required for analysis and the high cost of the equipment needed for automated or even manual PCR analysis can prohibit the clinical use of this method of nucleic acid analysis. The current use of PCR technology at the point-of-care and in developing countries is minimal. In these settings, the labor, skill, and expensive equipment required for PCR are larger barriers to full utilization of the technology.

There exists the need to overcome the barriers to using nucleic acid analysis. By minimizing the number of steps (and thus the time and skill required to perform a PCR reaction), and by reducing the instrumentation and costs per test, the present invention aims to expand the use of PCR technology to the clinic and developing countries.

### SUMMARY OF THE INVENTION

The present invention provides an apparatus for thermocycling a reaction mixture in a continuous flow that includes a lateral flow device having proximal and distal ends and an instrument for receiving the lateral flow device. The lateral flow device includes (a) a sample reservoir located at the proximal end of the lateral flow device; (b) a wicking pad located at the distal end of the lateral flow device; and (c) a porous membrane, having a proximal amplification zone and a distal detection zone. The porous membrane is located between and contacting the sample pad and the wicking pads, thus connecting the sample reservoir and the wicking pad. Preferably the lateral flow device is disposable. The instrument into which the lateral flow device is placed contains a first temperature block that has a plurality of stationary thermal zones. The temperature block is arranged to fit between the sample reservoir and the wicking pad and in contact with the porous membrane of the lateral flow device within the amplification zone.

The stationary thermal zones within the temperature block include a plurality of thermally conductive bars, a plurality of heaters, and a plurality of temperature controllers. Typically, each thermally conductive bar is at a different reaction temperature and there is one heater and one temperature controller for each thermally conductive bar. Each of the thermally conductive bars further contains a plurality of teeth, wherein the teeth are interdigitated and do not contact each other.

Generally, each temperature block contains at least one, more preferably two, and most preferably three thermally conductive bars. In one preferred embodiment the three thermally conductive bars include a first melting bar, a second extension bar, and a third annealing bar. The appropriate alignment of thermally conductive teeth for a thermocycling reaction is achieved by arranging the thermally conductive bars at 0°, 90°, and 180°.

The apparatus for thermocycling may also include an insulating cover that presses the porous membrane of the lateral flow device against the stationary thermal zones. The apparatus may also include a means for sealing the lateral flow device within the instrument. The means for sealing may be affixed to (or part of) the insulating cover. Alternatively, the means for sealing may be separate from the insulating cover.

The detection zone of the porous membrane includes a test line zone located distal to the amplification zone that captures and concentrates the amplicon product for visualization. In addition to the test line zone, the detection zone may also include a control line zone located distal to the test line zone that captures excess probe that has not bound amplicon product. Another feature that may be included in the detection zone with the test line zone, or both the test and control line zones, is a labeling zone located on the porous membrane between the amplification zone and the test line zone. The labeling zone contains amplicon-specific probe capable of recognizing, binding, and labeling amplicon product.

In preferred embodiments, the test line zone captures and concentrates amplicon product by means of a linear array of amplicon-capturing agents on the porous membrane. Similarly, the control line zone captures excess probe by means of a linear array of probe-capturing agent on the porous membrane. In a related aspect, the labeling zone contains the amplicon-specific probe on the porous membrane, but not adhered to the porous membrane, that labels the amplicon product so that the amplicon product may be visualized upon concentration in the test line zone.

The detection zone of the invention may further include a second temperature block having at least one stationary thermal zone. This second temperature block is useful for maintaining one or more regions of the detection zone at a different temperature. This feature is useful, for example if a nucleic acid hybridization were to be carried out in the detection zone.

The present invention further provides a method for amplifying nucleic acid by applying a nucleic acid amplification reaction mixture to the proximal end of a porous membrane and allowing the reaction mixture to travel toward the distal end of the porous membrane. The reaction mixture thereby flows through a plurality of proximal stationary thermal zones in contact with the porous membrane.

In certain aspects of the invention, the amplification step is followed by a concentration and detection step. Concentration and detection are preferably achieved by allowing the reaction mixture to travel through a distal detection zone on the porous membrane. Inherent to this step is allowing the nucleic acid to travel through at least one test line zone located within the detection zone that includes a linear array of amplicon-capturing agents on the porous membrane. In certain preferred embodiments, this step may further include allowing the reaction to travel through a control line zone having a linear array of a probe-capturing agent on the porous membrane. In other preferred embodiments, the step of concentrating and detecting may include allowing the nucleic acid to travel through a labeling zone region that contains amplicon-specific probes on the porous membrane that are available for binding to and flowing with the amplicon product to the test line zone (and optionally the control line zone).

In certain preferred embodiments, it may be desirable to assay the porous membrane for the presence of the probe in the detection zone. The presence of the probe indicates that the target nucleic acid was present in the sample and successfully amplified.

Finally, the invention provides a method for amplifying nucleic acid using the inventive thermocycling device. The method requires the steps of applying a nucleic acid amplification mixture to the sample reservoir and allowing the nucleic acid to travel through the amplification zone. In certain preferred embodiments, the method includes the step of concentrating and detecting the nucleic acid, the concentrating and detecting being achieved by allowing the nucleic acid to travel through the detection zone. As mentioned above, it may be desirable in certain embodiments to assay the porous membrane for the presence of the probe in the detection zone, the presence of the probe indicating presence and successful amplification of the target nucleic acid.

By “continuous flow,” as used herein, is meant that the movement of a reaction mixture for thermocycling is uninterrupted throughout the amplification process. For example, in the present invention, the reaction mixture travels in an uninterrupted fashion along a porous membrane.

The term “lateral flow device,” as used herein, refers to an apparatus that is designed to permit a continuous, lateral flow of a reaction mixture. The lateral flow device of the present invention permits lateral movement of a particular reaction mixture from the proximal end of the lateral flow device to the distal end of the lateral flow device without interruption.

By “sample reservoir,” as used herein, is meant a receptacle for the application of a reaction mixture onto the lateral flow device. For example, the sample reservoir of the present invention may be a tube into which the proximal end of the porous membrane is immersed to allow the reaction mixture to enter the porous membrane. Alternatively, the sample reservoir may be a pad located at the proximal end of the porous membrane and in contact with the proximal end of the porous membrane. A reaction mixture may be applied to the pad and absorbed into the pad. The reaction mixture subsequently leaches into the proximal end of the porous membrane by means of capillary action, wherefrom it will travel laterally along the porous membrane in continuous flow. The term “sample reservoir,” according to the present invention, is also meant to encompass the region encompassing the proximal end of the porous membrane onto which a reaction mixture can be directly applied.

By “wicking pad,” as used herein, is meant a piece of absorbent material that is placed in contact with the distal end of the porous membrane. The wicking pad acts to draw the reaction mixture applied to the proximal end of the porous membrane to the distal end of the porous membrane by capillary action. Exemplary wicking pads include any absorbent material, for example paper, such as nitrocellulose (e.g., S&S 3900 paper, Keene, N.H.).

The term “porous membrane” or “membrane,” as used herein, refers to a length of absorbent material along which a reaction mixture can travel by capillary action without hindrance. Typically, this requires that the absorbent material have a pore size of at least five microns. Examples of porous membranes include nitrocellulose, nylon, and polysolfone. A wide variety of materials that classify as porous membranes are commercially available.

By “temperature block,” as used herein, is meant a unit that provides the means for supplying at least one stationary thermal zone. More typically, the temperature block will provide the means for supplying a plurality of stationary thermal zones. Preferably, the temperature block has at least one flat surface for contacting the porous membrane.

By “stationary thermal zone,” as used herein, is meant a region that is fixed in position and capable of supplying a constant temperature. The stationary thermal zones of the present invention are composed of thermally conductive bars.

As used herein, the term “thermally conductive bar” is used to refer to a bar (e.g., of rectangular or square shape) made of a conductive material, for example aluminum. One bar is capable of conducting one reaction temperature. Each thermally conductive bar is provided with a heater and a temperature controller. In certain embodiments of the invention, each thermally conductive bar is further constructed to have a plurality of teeth that are interdigitated with the teeth of another thermally conductive bar or bars so that various stationary thermal zones are alternated.

By “heater,” as used herein, is used to refer to any means of supplying heat to a thermally conductive bar.

By “temperature controller,” as used herein, is used to refer to a device that regulates the specific temperature of the particular thermally conductive bar.

By “teeth,” as used herein, is meant an arrangement of rectangular prongs separated by a measured space. The teeth of the present invention are located along the length of the thermally conductive bar. According to the present invention, the teeth of the thermally conductive bar are typically in contact with the porous membrane. The width and spacing of the teeth is important for determining the length of time a reaction mixture, traveling from the proximal end of the porous membrane to the distal end of the porous membrane, will be in contact with a particular temperature provided by a particular thermally conductive bar. The width and spacing of the teeth depends in part on what purpose the teeth of that particular thermally conductive bar will serve., (i.e., denaturation, annealing, or extension) As but one example, the width of each tooth may be 2.5 mm and the spacing between each tooth may be 0.5 mm.

The term “proximal amplification zone” or “amplification zone,” as used herein, typically refers to the proximal region of the porous membrane within which the thermal cycles necessary for amplification occur. The amplification zone of the porous membrane is the region of the membrane in contact with the a temperature block that provides an alternating arrangement of a plurality of at least two stationary thermal zones. Preferably, the temperature block in contact with the amplification zone provides an alternating arrangement of three stationary thermal zones.

The term “distal detection zone” or “detection zone,” as used herein, refers to the distal region of the porous membrane within which processes necessary for concentration and detection of the amplicon occur.

The term “different,” as used herein, means not the same as. In particular, when different is used to refer to a different reaction temperature of a thermally conductive bar, the term different means that the temperature of one reaction bar is, for example, 95° C. and the temperature of another reaction bar is, for example, 55° C.

The term “melting bar,” as used herein, refers to a thermally conductive bar that is at a temperature sufficient for melting any double stranded nucleic acid molecule (i.e., 95° C.), for example, an RNA/DNA hybrid molecule, a double stranded DNA molecule, or a double stranded RNA molecule.

The term “extension bar,” as used herein, is used to refer to a thermally conductive bar that is at a temperature sufficient for a DNA polymerization reaction to proceed on a nucleic acid template using, for example, Taq polymerase. A typical extension bar temperature suitable for nucleic acid polymerization on a nucleic acid template is, for example 72° C.

The term “annealing bar,” as used herein, is used to refer to a thermally conductive bar that is at a temperature sufficient for annealing any two complementary single stranded nucleic acid molecules to generate a double stranded nucleic acid molecule, (e.g., a double stranded DNA molecule, a double stranded RNA molecule, or a hybrid RNA/ DNA molecule); typically as in annealing a primer to a target DNA molecule.

The term “insulating cover,” as used herein, means a lid that presses the membrane portion of the lateral flow device against the temperature block to achieve efficient heat transfer between the temperature block and the porous membrane. The insulating cover may, in addition, optionally be used to provide a means for sealing the lateral flow device within the instrument.

By “means for sealing,” as used herein, is meant a means for enclosing the lateral flow device within an instrument unit. The means for sealing may provide the function of reducing or preventing evaporation of the reaction mixture from the membrane during operation of the thermocycling device of the invention. In certain preferred embodiments, the means for sealing may be separate from the insulating cover.

By “instrument” is meant the apparatus for receiving the lateral flow device that includes the encasement; at least one temperature block that includes a plurality of stationary thermal zones, the temperature block being arranged to fit between the sample reservoir and the wicking pad and in contact with the porous membrane of the lateral flow device; a plurality of heaters; and a plurality of temperature controllers.

The term “test line zone,” as used herein, refers to a region of the porous membrane, preferably a cross section along the length of the membrane distal to the amplification zone, that includes an amplicon-capturing agent. The test line zone of the invention provides the means for detecting successful amplification by capturing the amplicon product within a narrow band of the porous membrane. Preferably, the captured amplicon is labeled (i.e., with an amplicon-specific probe) in such a way to permit visualization of the captured amplicon within the test line zone.

The term “control line zone,” as used herein, refers to a region of the porous membrane, preferably a cross section along the length of the membrane, that includes a probe-capturing agent. The control line zone, according to the invention, is preferably located distal to the test line zone, so that any probe that did not specifically bind to amplicon product is captured and detected in the control line zone. The amount of probe captured within the control line zone serves as a control for the amplification reaction. For example, if a relatively small quantity of probe is captured in the control line zone, compared to the test line zone, it is likely that the amplification reaction was a success. Alternatively, if a large quantity of probe is captured in the control line zone, compared to the test line zone, it is likely that the amplification reaction was unsuccessful. These results may indicate the presence or absence of target nucleic acid in the sample, respectively.

The term “labeling zone,” as used herein, refers to a region of the porous membrane, preferably a cross section along the length of the membrane, that includes an amplicon-specific probe. Preferably, the labeling zone is located distal to the amplification zone and proximal to the test line zone. According to the invention, as the reaction mixture leaves the amplification zone and enters the labeling zone, the amplified product, or “amplicon,” is bound by, and thereby labeled by, a probe that specifically recognizes the amplicon product.

The term “amplicon-capturing agent,” as used herein, refers to a means for binding to an amplified nucleic acid product. The amplicon-capturing agent is preferably adhered to the porous membrane within the test line zone, thereby trapping the amplicon within the test line zone so that the amplicon is concentrated within the test line zone and can be detected. According to the invention, an amplicon-capturing agent preferably binds to the amplicon directly. Exemplary amplicon-capturing agents may include a mouse anti-DNA antibody, an anti-DNA antibody (e.g., single strand specific), or a nucleic acid complementary to the amplicon.

The term “probe-capturing agent,” as used herein, refers to a means for binding to an amplicon-specific probe. The probe-capturing agent is preferably adhered to the porous membrane within the control line zone. Adherence of the probe-capturing agent to the porous membrane within the control line zone provides the means for trapping any un-bound amplicon-specific probe (i.e., a probe that did not bind amplicon product and become trapped within the proximal test line zone), thereby concentrating the amplicon-specific probe within the control line zone so that the amplicon-specific probe may be detected. Exemplary probe capturing agents include goat anti-mouse antibodies and anti-DNA antibodies that recognize both double stranded and single stranded DNA.

The term “amplicon-specific probe” or “probe,” as used herein, includes a detectable label conjugated to a molecule capable of binding an amplicon. Unlike the amplicon-capturing agent and the probe-capturing agent, the amplicon-specific probe is preferably not adhered to the porous membrane so that upon binding to the amplicon, the amplicon-specific probe is drawn, by capillary action, through the porous membrane to the test line zone (and optionally the control line zone). By way of example, one preferred amplicon-specific probe includes a colloidal gold particle (as the detectable label) conjugated to a mouse anti-DNA antibody. Of course the skilled artisan will recognize that any detectable label can be conjugated to any molecule capable of binding an amplicon to generate a amplicon-specific probe according to the invention.

### BRIEF DESCRIPTION OF THE DRAWING

FIG. 1 is a drawing that represents the thermally conductive bars that generate temperature zones. Panel A depicts the alignment for joining three thermally conductive bars, the bar in the center being the melting bar, the bar at the left being the annealing bar and the bar at the right being the extension bar. Panel B depicts an assembled configuration of three thermally conductive bars.

FIG. 2 is a drawing depicting a lateral flow device in contact with a temperature block. In this figure, both the amplicon-specific probe and the amplicon-capturing agent includes an antibody. Panel A represents the lateral flow device before completion of a thermocycling reaction. Panel B represents the lateral flow device after completion of a thermocycling reaction.

FIG. 3, represents a lateral flow device in contact with two temperature blocks. In this figure, the amplicon-specific probe includes an antibody and the amplicon-capturing agent includes an oligonucleotide. Panel A represents the lateral flow device before completion of a thermocycling reaction. Panel B represents the lateral flow device after completion of a thermocycling reaction.

FIG. 4 also represents a lateral flow device in contact with two temperature blocks. In this figure, the amplicon-specific probe includes an oligonucleotide and the amplicon-capturing agent includes an antibody. Panel A represents the lateral flow device before completion of a thermocycling reaction. Panel B represents the lateral flow device after completion of a thermocycling reaction.

### DESCRIPTION OF THE PREFERRED EMBODIMENTS

The present invention provides an apparatus and method for performing a thermocycling reaction for amplifying nucleic acid (i.e., (PCR)). In addition, the apparatus provides a means for concentrating and detecting the amplified nucleic acid product, or “amplicon,” without removing the sample from the apparatus.

A key feature of the invention is its reliance on the capillary effect of a porous membrane to draw a reaction mixture containing a sample and some or all of the amplification reagents through different zones along the membrane. One “zone” is the “amplification zone” 18 that includes alternating stationary thermal zones that heat or cool the reaction mixture as a function of its position within the membrane. Movement of the reaction mixture through a linear array of stationary thermal zones acts to perform the thermal cycles necessary for nucleic acid amplification. Another zone, referred to as the “detection zone” 9, contains means for concentrating and detecting the amplicon product while the amplicon product is still on the porous membrane. The combination of the amplification zone feature with the detection zone feature results in a device that can accomplish a complete amplification, concentration, and detection procedure very rapidly.

In traditional PCR, the vessel containing the reaction mixture, as well as the portions of the instrument containing and in contact with the reaction mixture, must be heated and cooled. Since the thermal mass of the vessel and the instrument are typically many orders of magnitude larger than the thermal mass of the reaction mixture, the time necessary for amplification is determined by the mass of the instrument and the vessel. In contrast, in lateral flow PCR, only the reaction mixture is heated and cooled. Moreover, the surface area to volume ratio of the membrane (and thus the sample contained within the membrane) is very large. This high surface area to volume ratio improves the thermal transfer rate. Temperatures in the reaction mixture will reach thermal equilibrium almost instantaneously upon contact with a particular stationary thermal zone. In the present invention, the rate at which amplification occurs is limited only by the reaction kinetics. The present invention thereby makes it possible to increase the amplification rate (and thus decrease the time required for amplification) by orders of magnitude compared to most traditional PCR amplification protocols currently available.

The detection zone feature of the invention contributes to the speed in which a reaction mixture, once amplified, is detected. The detection zone feature imparts a distinct advantage in that it provides one step concentration and detection, without having to transfer the reaction mixture to another vessel or modify the sample in any way. The skilled artisan will appreciate that the steps of concentrating and detecting amplicon products are easily coupled to the steps of amplifying because each of these steps occurs on the same porous membrane. This feature eliminates the number of steps and thus the amount of time and level of skill required to complete a thermocycling and detection procedure. In addition, in preferred embodiments, the results (presence or absence of amplicon) are easily read by eye, obviating the need for additional expensive and complicated instrumentation. The advantages provided by the detection zone feature clearly make the thermocycling device of the present invention practical for use in the field, in the doctor's office, in emergency rooms, and in developing countries.

Another advantage provided by the invention is the minimal amount of power required to operate the thermocycling device. Since only the reaction mixture is thermally cycled, very little heat is required. Preferably, the instrument uses less than 50 watts, more preferably less than 30 watts of power, and most preferably less than 20 watts of power. Further miniaturization is likely to further reduce the required power to a few watts (e.g., 1-10 watts), making battery and/or solar power instruments practical. Such instruments will contribute to the widespread applications in the field (i.e., outside the conventional laboratory) and in developing countries.

As mentioned above, a major advantage of the present invention is its simplicity of operation and low cost. The thermocycling device of the present invention has no moving parts. Movement of the sample through different temperature zones does not require pumping, as in some amplification devices, but is achieved by capillary action. The cost of the instrument, including the temperature blocks, should be considerably lower than currently available technologies. The lateral flow device, including a plastic housing is also relatively less expensive. In certain preferred embodiments, parts of the instrument, such as the lateral flow device, may be low enough in cost to be disposable.

Another advantage of the present thermocycling device is that is reduces the risk of cross-over contamination because only the lateral flow device portion of the apparatus comes in contact with the reaction mixture and the lateral flow device is disposable.

Various processing steps can be easily added to the lateral flow PCR process with little or no input from the user. For example, Mg²⁺ can be incorporated into a sample reservoir (e.g., a sample pad) and eliminated from the reaction mixture so as to minimize the formation of primer-dimers before the amplification starts. The results should be similar to the well known “Hot Start” approach (Qiagen #203,443). As but another example, one may use a nonthermophilic polymerase (i.e., a polymerase that adheres to the porous membrane). By binding the polymerase to the membrane in the amplification zone, one may omit the polymerase from the reaction mixture. In addition, since in this embodiment the polymerase is attached to the membrane and thus will not be traveling through multiple stable thermal zones set at denaturing temperatures, a thermoliable polymerase may be used.

The simplicity and ease of use of the inventive thermocycling device provide the additional advantage of eliminating the need for the expensive equipment currently required for performing automated, or even manual, thermocycling reactions. The high cost of such machinery is often prohibitive to performing thermocycling reactions for nucleic acid analysis in clinical laboratories, at the point-of-care, or in developing countries.

Just as the lateral flow immunochromatographic strip tests (like the home pregnancy tests) have made tests for specific proteins and antibodies practical for use in doctor's offices, emergency rooms, developing countries, and at home, the low cost and ease of use of the present thermocycling device makes nucleic acid analysis practical for a variety of setting that were previously prohibitive to nucleic acid analysis. Typically, one sample is loaded per membrane, however, multiple sets of primers can be added to the reaction mixture so that more than one DNA sequence can be detected in one test.

In one preferred embodiment, the thermocycling device of the present invention is applicable to a variety of military operations. The present invention may be used, for example, to detect biological warfare agents in the field, (such as a pathogenic virus or bacteria). In a related aspect, the present invention may be used to detect DNA taggants in the field. A taggant is a non-reactive substance added to an explosive for identification purposes. If the explosive is used for unlawful purposes, the origin of the explosive can be determined by analyzing the DNA taggant.

In an alternative embodiment, as mentioned above, the present invention may expand the use of the amplification reaction in the clinic and in developing countries. For example, the present invention may provide low cost genetic screening to clinics, particularly clinics in developing countries. In another preferred embodiment, competitive assays may be developed to detect point mutations, making the inventive system practical for genetic screening. In yet another preferred embodiment, the present invention is used to screen pathogens in blood supplies (e.g., to screen donated blood for the presence of HIV or hepatitis).

Alternatively, the thermocycling device of the present invention is used to screen for, or detect cancer (e.g., skin cancer) caused by particular known mutations in a patient's DNA.

In another related embodiment, the apparatus and method of the present invention may be used for detection of bacterial and viral disease causing agents in the doctor's office. This may lead to better, cheaper, and more widely available tests for venereal diseases, streptococcus, HIV, malaria, hepatitis, denuge, etc., which not only identify the specific infective agent, but identify the specific strain of the infective agent. As mentioned above, an automated clinical lab machine is not required for operation of the present invention. Thus, due to the low cost and low infrastructure requirements of the present thermocycling device, tests for pathogenic infection will be practical for use in the clinic and in developing countries.

In a related aspect, the thermocycling device of the present invention is applicable for detection of veterinary and horticultural disease agents in the field. In another related aspect, the inventive thermocycling device may be used to test for food safety in the field, for example, for inspecting the food in the kitchen of a food service provider for contamination (e.g., hepatitis contamination).

Lateral Flow Thermocycling Device: Amplification

The present invention provides an apparatus for thermocycling a reaction mixture in a continuous flow as the reaction mixture travels through a porous membrane with thermally controlled zones. Preferably, the apparatus provides a means for performing the polymerase chain reaction or other thermal cyclic amplification procedure well known in the field.

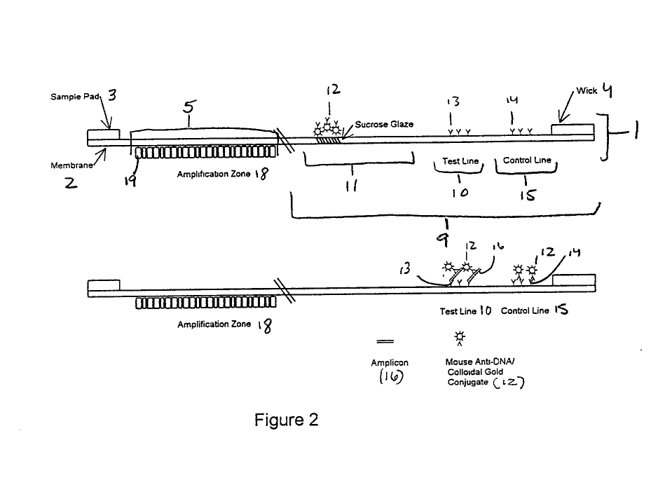

The thermocycling apparatus of the invention includes a lateral flow device 1 and an instrument for receiving the lateral flow device (see, FIG. 2). The lateral flow device 1 includes a porous membrane 2 with proximal and distal ends, a sample reservoir 3, and a wicking pad 4.

A central component of the lateral flow device is the porous membrane. The porous membrane is located between and contacting the sample reservoir and the wicking pad and provides the means by which the reaction mixture is drawn through the fixed amplification and detection zones by capillary action. The porous membrane is preferably a long, thin strip (approximately 100 mm long and 3-5 mm wide) of absorbent material, preferably nitrocellulose paper (e.g., Costar # 9420, Cambridge, Mass.). However, any material (nylon, polysolfone, etc. with a porosity of 5-15 μm can be used in the present invention. The membrane may be modified by blocking with a solution such as a 1% solution of polyvinyl alcohol (Sigma #P8136, St. Louis, Mo.), or Polyvinyl Pyrrolidone (Sigma #PVP-10, St. Louis, Mo.) to prevent components of the reaction mixture from sticking.

The sample reservoir 3 is located at the proximal end of the porous membrane 2. In one preferred embodiment, the sample reservoir is merely the proximal region of the porous membrane designated to receive the reaction mixture. In another preferred embodiment, the sample reservoir is a vessel that contains the reaction mixture into which the proximal end of the porous membrane is dipped. This allows the reaction mixture to be absorbed into the proximal end of the porous membrane by capillary action. In yet another preferred embodiment, a sample pad 3 is placed in contact with the proximal end of the porous membrane. The reaction mixture may be applied to the sample pad and allowed to leach through the sample pad into the porous membrane via capillary action. As but one example, a preferred material for use as a sample pad is S&S Accuflow G (Keene, N.H.). The skilled artisan will appreciate that many absorbent materials may be suitable for use as a sample pad.

The wicking pad 4 is located at the distal end of the porous membrane 2. Preferably, the wicking pad 4 is composed of a piece of absorbent material (e.g., typically paper, for example S&S 3900 paper, Keene, N.H.) and contacts the distal end of the porous membrane 2.

As mentioned above, the thermocycling apparatus also includes an instrument into which the lateral flow device is placed. The instrument preferably contains the lateral flow device on at least three sides. In addition, the instrument contains at least one temperature block 5. The temperature block 5 is placed within the instrument such that upon receiving the lateral flow device, the temperature block fits between the sample reservoir and the wicking pad and is in contact with the porous membrane. In certain preferred embodiments, the temperature block is fixed to an insulating lid (see, below).

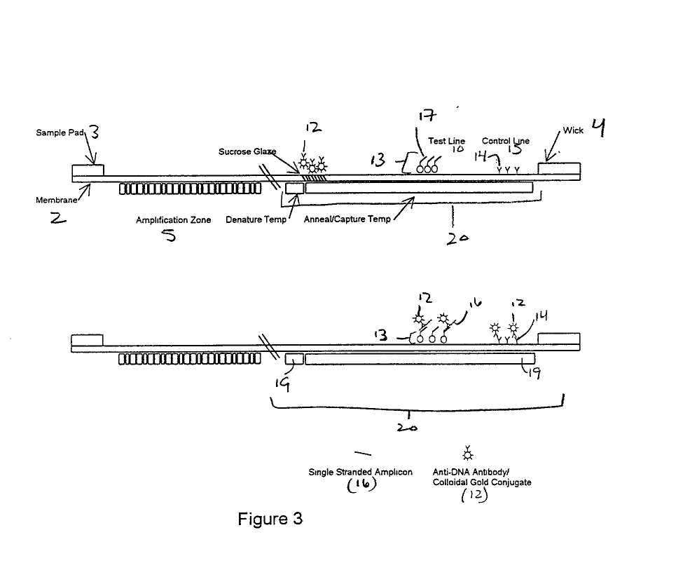

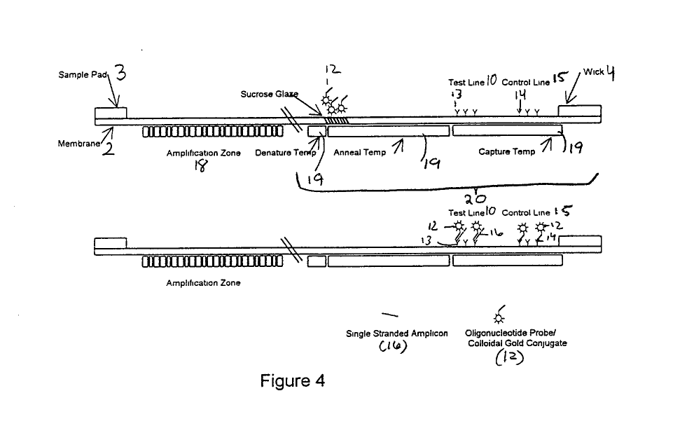

In preferred embodiments, the amplification device has a proximal amplification zone 18 and a distal detection zone 9 on the porous membrane 2. As part of this embodiment, it may be desirable to include a second temperature block in the instument (see FIGS. 3 and 4). In one preferred embodiment, a second temperature block 20 is placed in contact with the lateral detection zone 9 of the porous membrane 2. For example, in one particularly preferred embodiment, the first temperature block is placed in contact with the amplification zone of the porous membrane and the second amplification zone is placed in contact with the detection zone (discussed in further detail below).

The temperature block 5 or 20 of the invention includes at least one stationary thermal zone 19 that is fixed in position and temperature. More preferably, the temperature block will provide a plurality of stationary thermal zones in a linear array. The linear array of stationary thermal zones within the temperature block is arranged in a strip that matches the dimensions of the porous membrane. In order to achieve this arrangement, the temperature block perferably has at least one flat surface for contacting the porous membrane.

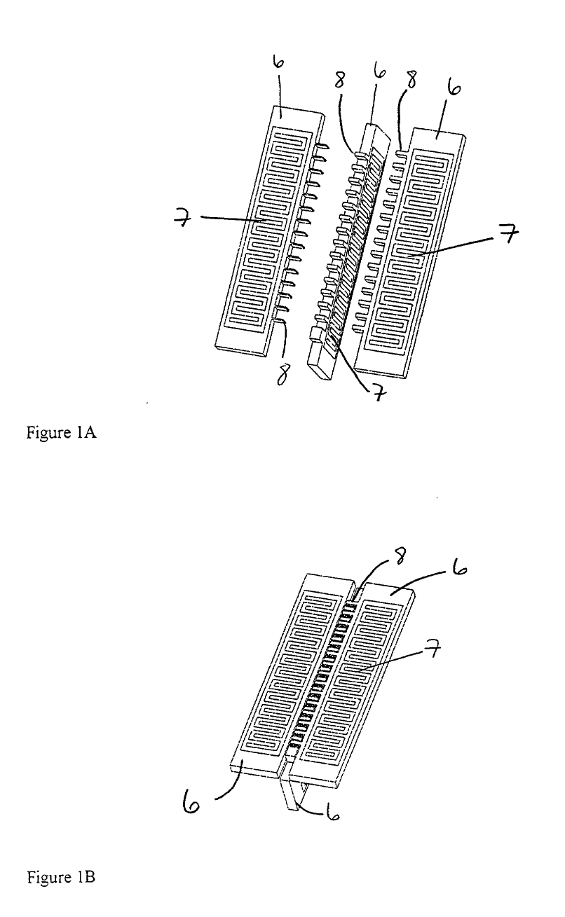

In one embodiment of the instrument, the temperature block 5 is composed of at least one, more preferably two, most preferably three thermally conductive bars 6 (one for each reaction temperature of the thermocycling reaction) (FIG. 1). The number of thermally conductive bars determines the number different stationary temperature zones in a particular lateral flow device. For example, in preferred embodiments of the present invention, a first thermally conductive bar, a “melting bar,” maintains a temperature capable of melting or denaturing double stranded nucleic acid (e.g., 95° C.), preferably double stranded DNA. A second thermally conductive bar, an “annealing bar,” maintains a temperature (e.g., 55° C.) that promotes annealing of single stranded primers of nucleic acids to single stranded target nucleic acid molecules. A third thermally conductive bar, an “extension bar,” maintains a temperature (e.g., 72° C.) that is conducive to the polymerase extension reaction that drives the nucleic acid amplification process. According to the present invention, the thermally conductive bars may be any shape (e.g., square, rectangular). Preferably, the thermally conductive bars are rectangular. In addition, the thermally conductive bars may be made of any thermally conductive material (e.g., copper, titanium, or aluminum). Preferably the thermally conducive bars are made of aluminum.

In order to maintain the temperature of the thermally conductive bar, each thermally conductive bar includes a heater 7 and a temperature controller. A variety of heaters and temperature controllers are known in the art. For example, a Minco 02-HK5166R20.5L12B heater and a Minco #16-CT198-1007R30.0L1 temperature controller (Minneapolis, Minn.) can be attached to the thermally conductive bar. Of course, any heater capable of maintaining a constant temperature of a thermally conductive bar can be used in the invention.

In order to provide a plurality of stationary thermal zones to the lateral flow device, teeth 8 are machined onto the thermally conductive bars (see, FIG. 1). The geometry of the bars is such that the teeth do not contact each other. This feature maintains thermal isolation between the bars. The bars are mounted at 0°, 90°, and 180° so that the teeth are interdigitated (see, FIG. 1). Preferably the teeth of each thermally conductive bar alternate consecutively with one another (for example tooth 1, tooth 2, tooth 3, tooth 1, tooth 2, teeth 3, etc.). This generates the linear array of teeth (and thus the linear array of stationary zones) required to align with the porous membrane strip of the lateral flow device. According to the design of the apparatus, during amplification the reaction mixture proceeds through a linear array of stationary thermal zones. These stationary thermal zones are provided by the teeth of the thermally conductive bar that are aligned with and in contact with the porous membrane.

The number of teeth on each bar determines the number of thermal cycles for the reaction. In preferred embodiments, any number of teeth can be machined onto the thermally conductive bars. In a typical example, 15 teeth are machined onto the thermally conductive bar, representing 15 thermal cycles. The width of the teeth determines the duration the reaction mixture is held at a particular temperature. For example, the flow rate of the reaction mixture through the lateral flow device is approximately 5 mm/minute. A tooth width of 5 mm would maintain one reaction temperature for approximately one minute.

The skilled artisan will appreciate that the number and dimensions of the teeth can be altered to fit the requirement of any thermocyclic reaction. As but one example, in a thermocycle requiring 15 cycles through three different temperature zones (a melting zone, an annealing zone, and an extension zone), each thermally conductive bar will have 15 teeth. The width of the teeth on the melting bar and extension bar may be, for example, 2.5 mm so that the reaction will remain in those zones for approximately 30 seconds. The first tooth of the melting bar may be twice as wide so that the initial melting time will be twice as long. The teeth on the annealing bar may be, for example, 0.8 mm so that the reaction mixture will remain in that zone for approximately 10 seconds (see, FIG. 1).

In certain preferred embodiments, the lateral flow device is placed on the instrument and covered with an insulating cover that presses the membrane portion of the lateral flow device against the thermal zones of the instrument to achieve efficient heat transfer. As part of (or in addition to) an insulating cover, a means for sealing the lateral flow device may be applied to the instrument. The means for sealing functions to enclose the lateral flow device and/or to reduce or prevent evaporation of the reaction mixture from the membrane during operation of the thermocycling device.

Lateral Flow Thermocycling Device: Concentration and Detection

After the reaction mixture travels through the amplification zone, the reaction mixture travels over a detection zone located on the same porous membrane as the amplification zone, distal to the amplification zone. (Of course, in certain preferred embodiments, it may be desirable for the lateral flow device to contain only an amplification zone.)

The basic premise of the detection zone is that as the amplicon products are immobilized and concentrated in the detection zone through ligand-mediated trapping of labeled amplicon products in a region of the detection zone called the test line zone 10. Accumulation of the amplicon products in the test line zone results in a high contrast signal indicating the presence of the target DNA sequence in the starting sample.

In preferred embodiments, an amplicon-capturing agent 13 is immobilized on the membrane within the test line zone. In one preferred embodiment, this amplicon-capturing agent will trap the nucleic acid product as it moves through the detection zone by reacting specifically with the nucleic acid itself (i.e., an anti-DNA antibody or an oligonucleotide complementary to the amplicon). In an alternative preferred embodiment, this amplicon-capturing agent is an antibody that will trap the nucleic acid product by reacting specifically with a recognizable ligand that has been incorporated into the amplicon (preferably a DNA amplicon) during the amplification reaction. According to the present invention, one or more ligands may be incorporated into the amplicon. The skilled artisan will appreciate that a wide variety of primary antibodies are available in the art that recognize a variety of ligands.

According to this aspect of the invention, the nucleic acid, preferably DNA, will generally be amplified using a mixture of modified deoxynucleoside triphosphates (dNTPs), which are the building blocks of DNA polymerization. There are four dNTPs that are assembled in a specific sequence that defines the DNA fragment of interest. There are several biochemical ligands that are particularly preferred for use in the present invention. For example, dNTPs may be tagged with immunogenic hapten groups, which include biotin, dinitrophenol, acetylamino fluorene analogs, digoxigenin, as well as sulphonated and brominated pyrimidines, although any ligand available in the art may be easily adapted for use in the present invention.

In order to detect the amplicon product immobilized in the test line zone, one may use an amplicon-specific probe. As mentioned above, the amplicon-specific probe 12 provides a means for visualizing an amplicon product at the end of amplification cycling. The amplicon-specific probe includes a detectable label conjugated to a molecule capable of binding the amplicon (amplicon-binding molecules). Any of a variety of methods and ligands, such as those described above, may be used to link the label to the amplicon-binding molecule. It is particularly preferred that the label be visible to the naked eye. One particularly preferred detectable label is a colloidal gold particle. Of course, other opaque particles may be used in the invention, for example, a latex particle. Both colloidal gold and latex particles are visible at low concentrations without instrumentation. Other detectable labels include horse red blood cells and graphite particles.

Preferred amplicon-binding molecules of the invention include molecules capable of recognizing and specifically binding the amplicon product. According to the invention, such a molecule may be a primary antibody. A preferred primary antibody is an antibody that binds double stranded DNA. One particularly preferred primary antibody is the mouse-anti DNA antibody (Roche part # 980 986, Indianapolis, Ind.). In another preferred embodiment, the primary antibody is specific for any ligand (such as those described above) that is incorporated into the amplicon during the amplification reaction. The incorporated ligand will thus provide the means by which to label the amplicon. For example, a colloidal gold particle may be conjugated to an antibody specific for an immunogenic hapten group into incorporated the amplicon.

Alternatively, a preferred amplicon-binding molecule is an oligonucleotide complementary to the amplicon product. According to the invention, the oligonucleotide may be conjugated to any label. In a particularly preferred embodiment, an oligonucleotide is conjugated to a colloidal gold particle and used as an amplicon-specific probe in the present invention.

In order to use the amplicon-specific probe to detect the amplicon concentrated in the test line zone, one may use a variety of standard assays well known in the art (see Ausubel et al., Current Protocols in Molecular Biology, Greene Publishing Associates, N.Y., V. 1&2, 1996 and Sambrook et al. Molecular Cloning: A Laboratory Manual, Cold Spring Harbor Press, N.Y., incorporated herein by reference.). For example, if the amplicon-specific probe includes an antibody, one may carry out the steps of a typical Western blotting procedure. To give but a few examples of the present embodiment, an amplicon which has incorporated one of the hapten immunogens can be retained at the test line zone by an antibody that is bound to the membrane in the test line zone and specific for that particular hapten immunogen. Colloidal gold particles conjugated to a hapten specific secondary antibody can then be used to detect the concentrated amplicon in the test line by carrying out the steps of a typical Western blot procedure. Of course, it will be appreciated that any calorimetric western blotting procedure available in the art may be used to detect the concentrated amplicon in place of a colloidal gold label.

Alternatively, if an amplicon-specific probe is being used to detect biotin labeled DNA, a streptavidin-conjugated colloidal gold amplicon-specific probe may be used. In yet another preferred embodiment, if the amplicon-specific probe includes an oligonucleotide conjugated to a label, the amplicon-specific probe may be hybridized to the amplicon. In prefered embodiments, the amplicon is denatured after exiting the amplication zone by traveling through the proximal region of the detection zone in contact with a stationary thermal zone set at a denaturing temperature. Thereafter, the sample may enter the region of a second stationary zone set at an annealing temperature.

In other preferred embodiments, the amplicon product is first labeled with an amplicon-specific probe prior to concentration and detection at the test line zone. Labeling of the amplicon product can be achieved by a variety of means. In one preferred embodiment, the reaction mixture itself contains amplicon-specific probe that binds to the amplicon product. For example, as the reaction mixture proceeds through the amplification zone, amplicon products are generated. As the amplicons are generated, the amplicon-specific probe in the reaction mixture binds to the amplicon product. The labeled amplicon product now continues to travel through the amplification zone by capillary action to the detection zone.

Alternatively, instead of providing the amplicon-specific probe in the reaction mixture, the detection zone may include a labeling zone 11. The labeling zone is a region of the porous membrane within the detection zone located proximal to the test line zone that contains amplicon-specific probe. To prevent the amplicon-specific probe from binding to the membrane, a sucrose glaze is applied to the membrane. The sucrose glaze is created by applying a 5-30% sucrose solution to the labeling zone and then drying the membrane. In certain preferred embodiments, the composition includes blocking reagents, (e.g., to prevent the amplicon-specific probe from binding to the membrane). Thus, in preferred embodiments the amplicon-specific probe is not bound to the porous membrane so that upon binding to the amplicon in the labeling zone, the amplicon-specific probe is drawn, by capillary action, through the porous membrane to the test line zone.

Of course the skilled artisan will recognize that any detectable label may be conjugated to any molecule capable of binding an amplicon to generate a amplicon-specific probe according to the invention.

Whether the amplicon-specific probe is provided in the reaction mixture, in the labeling zone, or after the amplicons are concentrated at the test line zone, according to the invention, the detection aspect of the invention relies on the fact that as the amplicons (and thus the amplicon-specific probes) are concentrated in the detection zone, resulting in a visible spot on the line.

In certain preferred embodiments, the detection zone may include a control line zone 15, a region of the porous membrane located distal to the test line zone. The purpose of the control line zone is to capture any amplicon-specific probe that is not captured by the test line zone. This provides a measure of the success of the amplification reaction. For example, if the amplicon-specific probe is captured in the test line zone, this indicates that the thermocycling reaction was successful and amplicon products were generated. Successful amplification indicates the presence of the target nucleic acid in the sample. Alternatively, if the amplicon-specific probe is captured in the control line zone, but not in the test line zone, this indicates that the thermocycling reaction did not successfully generate amplicon-specific product and the target DNA was not present in the sample.

The porous membrane of the control line zone has bound to it a probe-capturing agent. The probe-capturing agent can be any molecule capable of binging to, and trapping the amplicon-specific probe on, the porous membrane. If the amplicon-specific probe includes a protein component, a particularly preferred probe-capturing agent is an antibody (e.g., a goat anti-mouse antibody). In contrast, if the amplicon-specific probe includes a nucleic acid component, for example, an oligonucleotide, an antibody that recognizes single stranded DNA or an antibody that recognizes both single stranded and double stranded DNA may be used as the probe-capturing agent. Alternatively, if the amplicon-specific probe includes an oligonucleotide, a complementary oligonucleotide may be used as the probe capturing agent.

A Method of Amplifying Nucleic Acid

In PCR, amplification of nucleic acid is achieved by cycling the reaction mixture through at least two, more preferably three temperatures, for example, the melting temperature (e.g., 95° C.), the annealing temperature (e.g., 55° C.), and the extension temperature (e.g., 72° C.). In a conventional PCR apparatus, a sample tube containing the reaction mixture is heated and cooled over time as a batch in a cyclic fashion. In contrast, the present invention maintains stationary thermal zones along a porous membrane through which the reaction mixture travels by capillary action.

The present invention provides a method for amplifying nucleic acid that includes the steps of (1) applying a nucleic acid amplification reaction mixture to the proximal end of a porous membrane; and (2) allowing the reaction mixture to travel toward the distal end of the porous membrane. Step 2 requires that the reaction mixture travels through an amplification zone that includes a plurality of proximal stationary thermal zones in contact with the porous membrane. These stationary thermal zones are preferably arranged in a linear array such that a reaction mixture traveling through the porous membrane will encounter multiple cycles of melting, annealing and extending temperatures required for nucleic acid amplification.

The method of the invention optionally includes concentrating and detecting the amplified nucleic acid. The step of concentrating and detecting is accomplished by allowing the reaction mixture, once it traverses the amplification zone, to travel through a distal detection zone on the porous membrane. Within the detection zone, the reaction mixture will encounter a test line zone that includes a linear array of amplicon-capturing agents fixed to the porous membrane. The amplicon-capturing agents are allowed to bind and thereby concentrate the amplified nucleic acid so that the amplified product may be visualized.

In another preferred embodiment, the step of concentrating and detecting may optionally include allowing the amplified nucleic acid to travel through a control line zone located within the detection zone distal to the test line zone. The control line zone includes a linear array of a probe-capturing agent on the porous membrane. The probe-capturing agents are allowed to bind to, and thereby concentrate any amplicon-specific probe that has not bound amplicon.

In yet another preferred embodiment, the step of concentrating and detecting optionally includes allowing the nucleic acid to travel through a labeling zone located between the stationary thermal zones and the test line zone. The labeling zone includes amplicon-specific probes on the porous membrane.

Alternatively, in the absence of a labeling zone, the amplicon-specific probe may be included in the reaction mixture. The amplicon-specific probe is allowed to bind the amplicon and subsequently allowed to bind to a amplicon-capturing agent in the test line zone, or to bind to a probe-capturing agent in the control line zone, where visualization may occur. In an alternative preferred embodiment, if the amplicon specific probe is not provided in a labeling zone or in the reaction mixture, the method of concentrating and detecting may instead include a step of assaying the porous membrane for the presence of the probe in the detection zone. Such assays, such as Western blot and hybridization assays, are described herein. The presence of the probe in the test line zone indicates successful amplification of the nucleic acid.

Method of Using the Lateral Flow PCR Device

The present invention provides a method for amplifying nucleic acid using the thermocycling apparatus described herein. In preferred embodiments, the method includes the steps of (1) applying a nucleic acid amplification mixture to the sample reservoir; and; (2) allowing the nucleic acid to travel through the amplification zone. In addition, the method may further include a step of concentrating and detecting the amplified nucleic acid by allowing the amplified nucleic acid to travel through the detection zone. In certain aspects, the method may further include assaying the porous membrane for the presence of the amplicon in the detection zone using an amplicon-specific probe. Visualization of the amplicon-specific probe indicates successful amplification of nucleic acid.

The present invention can be further understood through consideration of the following non-limiting Examples.

### EXAMPLES

### Example 1

### Non-specific Concentration and Detection

FIG. 2 depicts a non-specific concentration and visualization scheme for the detection of amplified nucleic acid products. In this example, positive detection of amplicon 16 relies on the specificity of the amplification process to determine the specificity of the test. In FIG. 2, panel A, the test is created by binding an anti-DNA antibody 13 (Roche part#980 986, for example) to the nitrocellulose membrane 2 at the test line zone 10 of the membrane 2 and a goat anti-mouse antibody 14 is bound to the membrane at the control line zone 15. An amplicon-specific probe 12 is constructed by conjugating a mouse anti-DNA antibody (Roche part #980 986, for example) to a colloidal gold marker. The amplicon-specific probe 12 is deposited on the membrane 2 (with blocking reagents) on the labeling zone 11 on a sucrose glaze to prevent the probe 12 from binding to the membrane 2 (Harvey et al., Guide to diagnostic rapid test device components 1999, Schlecher & Schrell, Keene N.H.).

The reaction mixture, including the sample, is applied to the sample reservoir. In this example, the sample reservoir is a sample pad 3. As the reaction mixture flows through the amplification zone 18 the target DNA, if present, is amplified. As the reaction mixture flows to the sucrose glaze of the labeling zone 11 it mixes with the mouse anti-DNA/colloidal gold conjugate probe 12 and is carried toward the distal end of the membrane 2 to the test line zone 10. During this process, the anti-DNA gold conjugate probe binds to the amplicon product 16. As the complex flows into the test line zone it is immobilized by the anti-DNA antibody (amplicon-capturing agent 13) bound to the membrane and a visual line develops due to concentration of the colloidal gold particles. Any probe that is not bound to the amplicon 16 travels downstream to the control line zone where it is captured, and thereby concentrated by the goat anti-mouse antibody. Two lines will appear (in the test line zone and the control line zone) (see, FIG. 2B) if the target DNA was present in the sample and was amplified. Only one line will appear (in the control zone) if the target DNA was not present in the sample and thus was not amplified.

### Example 2

### Specific Concentration and Non-specific Detection

FIG. 3 depicts a specific concentration and visualization scheme for the detection of amplified products 16. This example does not completely rely on the specificity of the amplification process since only amplicons with sequences homologous to the oligonucleotide 17 in the amplicon-capturing agent 13 are captured. In this example (FIG. 3A), the test is created by binding an oligonucleotide probe 13 to the nitrocellulose membrane 2 at the test line zone 10 of the membrane 2 and a goat anti-mouse antibody probe-capturing agent 14 to the membrane 2 at the control line zone 15. A probe 12 is constructed by conjugating a mouse anti-DNA antibody (Roche part #980 986 for example) to a colloidal gold marker. The probe 12 is deposited onto a sucrose glaze on the labeling zone 11 of the membrane 2 (with blocking reagents) to prevent the probe 12 from binding to the membrane 2.

The lateral flow device 1 is in contact with the second (distal) temperature block 20 containing two stationary thermal zones that function to maintain a denaturing temperature in the reaction mixture as it enters the detection zone 9 and maintain optimal temperature for probe hybridization/capture once the sample enters the labeling zone.

The reaction mixture, including the sample, is applied to the sample pad 3. As the reaction mixture flows through the amplification zone 18 the target DNA, if present, is amplified. As the reaction mixture flows to the sucrose glaze in the labeling zone 11 it mixes with the mouse anti-DNA/colloidal gold conjugate probe 12 and is carried toward the distal end of the membrane 2 to the test line zone 10. The anti-DNA gold conjugate probe 12 binds to the amplicons 16. As the complex flows into the test line zone 10 the amplicon 16 hybridizes with oligonucleotide containing amplicon-capturing agent 13 bound to the membrane 3 (U.S. Pat. No. 5,989,813) The amplicon 16 is immobilized by the amplicon capturing agent 13 by forming a complex and a visual line develops. Any probe which is not bound to the amplicon 16 travels downstream to the control line zone 15 where it is captured by the anti-DNA antibody 14. Two lines will appear (in the test line zone and the control line zone) (FIG. 3B) if the target DNA was present in the sample and amplified and only one line will appear (in the control zone) if the target DNA was not present in the sample and thus not amplified.

### Example 3

### Non-specific Concentration and Specific Detection

FIG. 4 depicts a specific concentration and visualization scheme for the detection of amplified products. This example does not completely rely on the specificity of the amplification process, since the probe 12 is specific to the target DNA molecule. In this example (FIG. 4A), the test is created by binding an anti-DNA (intercalated) antibody amplicon-capturing agent 13, which binds only to double stranded DNA (US Bio #D387, for example), to a nitrocellulose membrane 2 at the test line zone 10 of the membrane 2. An anti-DNA antibody probe capturing agent 14, which binds to both single and double stranded DNA (Roche part #980 986, for example) is bound to the membrane at the control line zone 15. A probe 21 is constructed by conjugating an oligonucleotide having a sequence homologous to the expected amplicon product to a colloidal gold marker. The probe 12 is deposited on the labeling zone 11 of the membrane 2 with blocking reagents to prevent the probe 12 from binding to the membrane 2.

The lateral flow device is in contact with a temperature block 20 that maintains a denaturing temperature in the reaction mixture as it enters the detection zone 9, and maintains an optimal temperature for probe hybridization and capture once the mixture enters the labeling zone. The temperature block depicted also has a third stationary temperature zone where that maintains a “capture temperature” that promotes binding at the test line and the control line.

The reaction mixture, including the sample, is applied to the sample pad 3. As the reaction mixture flows through the amplification zone 18 the target DNA, if present, is amplified. As the reaction mixture flows to the labeling zone 11, it mixes with the oligonucleotide/colloidal gold conjugate probe 12 and is carried down stream to the test line zone 10. During this process, the probe 12 hybridizes with amplicon 16. As the complex flows into the test line zone 10 the amplicon 16 is captured by the anti-DNA (intercalated) antibody 13. The complex is immobilized and a visual line develops. Any probe 12 that is not bound to the amplicon 16 travels downstream to the control line zone 15 where it is captured by the anti-DNA antibody probe capturing agent 14. Two lines will appear (in the test line zone and the control line zone) (FIG. 4B) if the target DNA was present in the sample and amplified and only one line will appear (in the control zone) if the target DNA was not present in the sample and thus not amplified.

What is claimed is:

1. An apparatus for thermocycling a reaction mixture in a continuous flow comprising:
a lateral flow device having proximal and distal ends, wherein said lateral flow device comprises
(a) a sample reservoir located at said proximal end of said lateral flow device;
(b) a wicking pad located at said distal end of said lateral flow device; and
(c) a porous membrane located between and contacting said sample reservoir and said wicking pad; and
an instrument for receiving said lateral flow device comprising a temperature block comprising a plurality of stationary thermal zones, said temperature block arranged to fit between said sample reservoir and said wicking pad and in contact with said porous membrane.

2. The apparatus of claim 1, wherein the lateral flow device is disposable.

3. The apparatus of claim 1, wherein said porous membrane comprises a proximal amplification zone and a distal detection zone.

4. The apparatus of claim 1, wherein said stationary thermal zone comprises a plurality of thermally conductive bars, a plurality of heaters, and a plurality of temperature controllers.

5. The apparatus of claim 4, wherein each of said thermally conductive bars contains a plurality of teeth.

6. The apparatus of claim 5, wherein said teeth are interdigitated and do not contact each other.

7. The apparatus of claim 4, wherein each of said thermally conductive bars is a different reaction temperature.

8. The apparatus of claim 4, wherein said thermally conductive bars comprise a melting bar, an extension bar, and an annealing bar.

9. The apparatus of claim 8, wherein said thermally conductive bars are arranged at 0°, 90°, and 180°, with respect to each other.

10. The apparatus of claim 1, further comprising an insulating cover that presses said porous membrane against said stationary thermal zones of said lateral flow device.

11. The apparatus of claim 1, further comprising a means for sealing said lateral flow device within said instrument.

12. The apparatus of claim 3, wherein said detection zone comprises a test line zone, wherein said test line zone is located distal to said amplification zone.

13. The apparatus of claim 3, wherein said detection zone comprises a test line zone and a control line zone, wherein said test line zone is located distal to said amplification zone and said control line zone is located distal to said test line zone.

14. The apparatus of claim 3, wherein said detection zone comprises a labeling zone and a test line zone, wherein said labeling zone is located on said porous membrane distal to said amplification zone, and wherein said test line zone is located distal to said labeling zone.

15. The apparatus of claim 3, wherein said detection zone comprises a labeling zone, a test line zone, and a control line zone, wherein said labeling zone is located distal to said amplification zone, wherein said test line zone is located distal to said labeling zone, and wherein said control line zone is located distal to said test line zone.

16. The apparatus of claim 12, 13, 14, or 15 wherein said test line zone comprises a linear array of an amplicon-capturing agent on said porous membrane.

17. The apparatus of claim 13 or 15, wherein said control line zone comprises a linear array of a probe-capturing agent on said porous membrane.

18. The apparatus of claim 14 or 15, wherein said labeling zone comprises an amplicon-specific probe on said porous membrane.

19. The apparatus of claim 3, wherein said detection zone further comprises a second temperature block comprising at least one stationary thermal zone.

20. A method for amplifying nucleic acid comprising,
applying a nucleic acid amplification reaction mixture to the proximal end of a porous membrane; and
allowing said reaction mixture to travel toward the distal end of said porous membrane, wherein said reaction mixture travels through a plurality of proximal stationary thermal zones in contact with said porous membrane.

21. The method of claim 20, further comprising concentrating and detecting said nucleic acid, said concentrating and detecting comprising allowing said reaction mixture to travel through a distal detection zone on said porous membrane.

22. The method of claim 21, wherein said concentrating and detecting further comprise allowing said nucleic acid to travel through a test line zone located within said detection zone, said test line zone comprising a linear array of amplicon-capturing agents on said porous membrane.

23. The method of claim 22, wherein said concentrating and detecting further comprise allowing said nucleic acid to travel through a control line zone located within said detection zone distal to said test line zone, said control line zone comprising a linear array of a probe-capturing agent on said porous membrane.

24. The method of claim 22 or 23, wherein said concentrating and detecting further comprise allowing said nucleic acid to travel through a labeling zone located between said amplification zone and said test line zone, said labeling zone comprising amplicon-specific probes on said porous membrane.

25. The method of claim 20, further comprising assaying said porous membrane for the presence of said probe in said detection zone, the presence of said probe indicating successful amplification of said nucleic acid.

26. A method for amplifying nucleic acid using the apparatus of claim 1, said method comprising,
applying a nucleic acid amplification mixture to said sample reservoir; and
allowing said nucleic acid to travel through said amplification zone.

27. The method of claim 26, further comprising concentrating and detecting said nucleic acid, said concentrating and detecting comprising, allowing said nucleic acid to travel through said detection zone.

28. The method of claim 26, further comprising assaying said porous membrane for the presence of said probe in said detection zone, the presence of said probe indicating successful amplification of said nucleic acid.
